(19) United States
(12) Patent Application Publication
(10) Pub. No.: US 2004/0045806 A1
(43) Pub. Date: Mar. 11, 2004

(54) Method and device for treating the surfaces of items

(76) Inventors: Willi Neff, Kelmis (BE); Franz-Josef Trompeter, Aachen (DE); Oliver Franken, Eschweiler (DE); Klaus Pochner, Russelsheim (DE)

Correspondence Address: Breiner & Breiner, 115 North Henry Street, PO Box 19290, Alexandria, VA, 22320-0290, US

(21) Appl. No.: 10/432,167

(22) PCT Filed: Nov. 28, 2001
(86) PCT No.: PCT/DE01/04484

(30) Foreign Application Priority Data
Nov. 29, 2000 (DE) 10059131.0

(51) Int. Cl. B01J 19/08

(52) U.S. Cl. 204/164; 422/186.05

(57) ABSTRACT

The present invention relates to a method and a device for treating the surface of objects, in particular strip material or deep-drawn material, in which the to-be-treated surface (2) of the object (1) is subjected to a barrier discharge which is generated between a first planar electrode (4) and a second planar electrode (5) in a discharge region (3) which is filled with a first gas or gas mixture, with a plasma-excited second gas or gas mixture which emits UV radiation being utilized as the second electrode (5).

A surface treatment of greater efficiency and less duration including complete sterilization at low temperatures are achieved with the present method and the corresponding device.

### TECHNICAL FIELD

The present invention relates to a method and device for treating the surface of objects, especially the surface of strip material or deep-drawn material. The to-be-treated surface of the object is subjected to a barrier discharge in a discharge region filled with a first gas or gas mixture, said barrier discharge being generated between a first planar electrode and a second planar electrode.

The treatment of surfaces, in particular, their cleaning, degermination, sterilization, disinfection, or activation plays a significant role in many technical fields. For instance, the surface of strip material for packing has to be degerminated or sterilized before use. Such degermination or sterilization can be carried out in an advantageous manner, for example, using the present method and the present device.

### STATE OF THE ART

Methods and devices for cleaning surfaces are disclosed in DE 41 13 524 A1 and EP 510 503 A2. In both instances, a high-power discharge tube is provided distinctly spaced from a to-be-cleaned substrate. In the first case, the substrate is photo-chemically altered by UV radiation for better attachment of the coating material. In the second prior-art example, the UV radiation forms radicals. The UV radiation is generated by a barrier discharge in a high-power discharge tube. Such a barrier discharge, also referred to as dielectrically impeded discharge or still discharge in the literature, occurs in a discharge region formed between two electrodes, of which at least one electrode is separated from the discharge region by a dielectric barrier, when the sparking voltage respectively the sparking power in the discharge region is exceeded. Depending on the pressure range and the composition of the gas, a homogenous plasma or thin charge channels, so-called filaments, which exist only for a few nanoseconds, form. Such barrier discharges release UV radiation of high intensity in the discharge region when a suited gas is employed so that such type devices can be used as high-power UV-emitters. However, to do this at least one of the electrodes as well as the dielectric must be permeable for UV-radiation.

DE 43 02 465 C1 describes a device in which one of the electrodes is formed by a voltage-excited plasma in a gas whose pressure is at least two magnitudes lower than the gas pressure in the discharge region. The gas of the voltage-excited plasma used as an electrode is enclosed in a chamber made of a dielectric material whose sides running perpendicular to the first electrode are provided with one or a multiplicity of electrodes for exciting this low-pressure plasma. The dielectric material of the chamber is permeable for UV-radiation and simultaneously forms the dielectric barrier in the discharge region. The gas in the chamber is selected in such a manner that it is permeable, in particular in the plasma-excited state, for the UV radiation generated in the discharge region. AN UV-radiation-permeable electrode is realized in this manner. The applications of the UV radiation generated in the discharge region described in this printed publication relate to inducing chemical reactions, exciting dyestuffs and homogenizing medium-pressure plasma and high-pressure plasma in lasers and in plasma-enhanced material deposition from the gas phase.

DE 43 32 866 C2 discloses a method and a device for treating the surface of objects, in which the to-be-treated surface of the object is subjected to a barrier discharge, which is generated between a first and a second planar electrode, in a discharge region filled with a first gas, with the to-be-treated strip material directly forming the dielectric barrier between one of the electrodes and the discharge region. In a further embodiment of the disclosed method, the object is placed outside the discharge region immediately adjacent to the second electrode designed as a grid electrode in such a manner that the barrier discharge can act through the grid electrode on the surface of the object. The direct action of the barrier discharge results in cleaning the surface as a consequence of plasma-chemical decomposition.

In another embodiment disclosed in this printed publication, the discharge region is formed between a first planar electrode and a gas-filled chamber made of an UV-radiation-permeable dielectric material. This device known from DE 43 02 465 C1 is operated as a UV emitter with the to-be-treated surface being impinged in this case outside the discharge region by the UV radiation passing through the second electrode. The action of this UV radiation generated in the discharge region similarly results, by means of photo-chemical processes, also in cleaning the irradiated surface.

Based on this state of the art, the object of the present invention is to provide a method and a device for treating the surface of objects which permits increasing the efficiency and accelerating the surface treatment process. In particular, the device and the method should permit quick degermination of surfaces, especially of strip materials, as well as complete sterilization which has not hitherto been achievable with UV treatment.

### D SCRIPTION OF THE INVENTION

The object of the present invention is solved with the method and the device according to claims 1 respectively 12.

Advantageous embodiments of the method and the device are the subject matter of the subclaims. Finally, claim 22 describes an alternative manner of operating the invented device for treating surfaces.

In the present method, the to-be-treated surface of the object is subjected to a barrier discharge, which is generated between a first and a second planar electrode, in a discharge region filled with a first gas or gas mixture. A plasma-excited second gas or gas mixture is utilized as the second electrode which emits the UV radiation. In particular, the second plasma-excited gas or gas mixture is preferably also excited via a barrier discharge. This two-step discharge, on the one-hand the barrier discharge of the first gas or gas mixture in the discharge region and on the other hand the discharge respectively the barrier discharge of the second gas or gas mixture, leads to efficient and rapid surface treatment. The direct action of the barrier discharge in the discharge region in which the object is placed or passed through results in a plasma-chemical surface treatment by means of radicals while simultaneously, by means of the plasma-excited second gas serving as the second electrode respectively the barrier discharge in this gas, an intensive UV radiation of the surface is achieved.

In this manner, the second electrode can be designed similar to the second electrode formed by a plasma-excited gas of DE 43 02 465 C1. This printed publication utilizes the homogenizing effect and the UV permeability of the electrode, whereas in the present method gases or gas mixtures, such as for example noble gases or noble gas halogenide mixtures are filled into the chamber provided for the second gas, and these gases or gas mixtures themselves effectively generate UV in the barrier discharge occurring in this chamber. This strong UV-radiating gas discharge simultaneously represents the second electrode for the barrier discharge of the discharge region acting directly on the to-be-treated surface. The second electrode, referred to in the following as plasma electrode, is thus separated from direct gas discharge on the surface of the object and can be operated in overpressure or in underpressure, for example at 500*10² Pa (500 mbar). The substantially closer and more direct UV exposure of the to-be-treated surface without any masking metal electrode and the simultaneous treatment by the second direct barrier discharge improve the efficiency of the surface treatment in particular the cleaning action or the degerminating action on the surface. Apart from the quality of a solely UV treatment, the additional plasma-chemical action permits complete sterilization and therewith the application in aseptic packaging at temperatures <70° C.

The plasma-excited second gas or gas mixture is preferably subjected to a pressure of at least 100*10² Pa (100 mbar). Strong and optimized UV and UV emission can be obtained by suited selection of this second gas or gas mixture. Particularly suited for this purpose are state-of-the-art excimer gases, such as for example Xe or KrCl.

The gas in the discharge region can be, for example, air or moist air under atmospheric pressure. Preferably however gases, gas mixtures or vapors which enhance the desired surface treatment are additionally introduced into the discharge region. Thus, for instance, degermination can be enhanced by means of various mechanisms favorable to degermination. An example is increasing the UV emission in the barrier discharge of the discharge region by introducing argon or nitrogen or by admixing hydrogen. Similarly, the influence of particle bombardment, e.g. ions, on the to-be-cleaned surface is increased by admixing light gases, such as for example hydrogen. An increase in the cleaning action, in particular the disinfection and sterilization of the surface by means of additional chemical respectively plasma-chemical oxidation is obtained by admixing oxidative acting gas components, such as for example oxygen, ozone, hydrogen, water vapor, hydrogen peroxide gas or vapor to the gas mixture in the barrier discharge of the discharge region. Moreover, admixing noble gases, such as for example helium or argon, permits homogenizing the barrier discharge. A uniform surface coverage of the gas discharge enhances cleaning, in particular sterilizing, the surface. The discharge region for the additional introduction of such gases can be designed tunnel-shaped in such a manner that the additionally introduced gases, gas mixtures or vapors displace the ambient air. The tunnel-shaped design is obtained by means of a suited geometric shape of the electrodes.

In another advantageous embodiment of the present method, the barrier discharge in the discharge region is excited in a pulsed manner in order to obtain greater density of the discharge filaments or in order to obtain a uniform gas discharge on the to-be-degerminated surface. This pulsed excitation, such as is known, for example, from DE 196 43 925 A1, whose disclosure content relating to pulsed excitation is included in the present patent application, occurs by means of applying steep voltage increases to the electrodes which raises the sparking field power of the discharge filaments. With voltage increases from 1 kV/μs on—with an atmospheric pressure better than 10 kV/ns—distinctly raises the uniformity of the filaments as well as the UV exploitation in both gas discharges. The improved surface coverage of the discharge filaments related herewith enhances the cleaning action and the efficiency.

Preferably, large surface, planar electrodes are employed in carrying out the present method so that a large surface is simultaneously impinged with the barrier discharge as well as with the UV radiation. An arrangement of a multiplicity of such type electrodes behind one another and/or side by side for covering a large surface offers advantages, in particular acceleration of the process.

In treating the surface of strip material, the strip material is preferably moved through the discharge region between the plasma electrode and the grounded electrode. Again a multiplicity of such pairs of electrodes can be placed in the transport direction of this strip material in order to be able to impinge a large surface simultaneously with barrier discharges as well as with UV radiation.

The present device is provided with a discharge region which is formed between a first planar electrode and a, preferably closed, chamber filled with a gas or gas mixture, with at least a first first-electrode-facing side of the chamber being made of an UV-radiation-permeable dielectric material. On a second side facing away from the first electrode, the chamber borders a further planar metal electrode or is closed by it and is filled with a gas emitting UV-radiation in a plasma-excited state. In order to operate the device, an alternating voltage respectively a pulsed voltage is applied to the first and to the further electrode which leads to sparking the two plasmas.

Thus, contrary to a device as those of DE 43 32 866 C2 or DE 43 02 465 C1, no electrode is provided at the side wall of the chamber perpendicular to the first or second side. The present device differs from the prior-art devices in that the chamber is filled with a gas emitting UV-radiation in a plasma-excited state and in that the gas in the chamber is under higher pressure. Preferably the pressure of the gas in the chamber is at least 100*10² Pa (100 mbar), but can, however, also be distinctly above this.

Preferably the first and the further electrode as well as the first and second side of the chamber are designed plane and in parallel to each other. The dielectric material of the chamber may be made, for example, of quartz glass. The second side of the chamber can either also be made of quartz glass or directly formed by the further electrode. Of course, the first electrode can also be designed as a plasma electrode, i.e. in the form of a plasma-excited gas in a corresponding chamber.

In order to treat non-plane objects, for example deep-drawn objects, the electrodes can also have a three-dimensional form corresponding to the shape of the objects.

The present device is suited, in particular, for flat respectively thin objects, because the distance between the first side of the chamber and the first electrode usually lies in a range between one and five millimeters so that only correspondingly thin materials can be led through this discharge region or into this discharge region. When treating the surface of strip material, the strip material is led continuously or stepwise through the discharge region while the two discharges are maintained. The discharge region has to, of course, be provided with openings on both sides for feeding the strip material.

The present method and the present device can be especially used for cleaning, degerminating, sterilizing, disinfecting or activating surfaces. A particularly advantageous application relates to degerminating strip packing material which can be carried out faster and more efficiently with the present method and the corresponding device. A further advantageous application relates to cleaning wafers, in particular extra-fine cleaning or degreasing. Treatment of foils or activation of the surface of foils can also be carried out advantageously with the present method and the corresponding device.

The present device can also be operated in a manner in which only the sparking voltage is applied at the first and at the further electrode, in which the plasma in the chamber sparks but not in the discharge region under atmospheric pressure. In this manner, a thin UV emitter without a masking wire mesh electrode is realized via which the to-be-treated surface is impinged in immediate proximity with UV radiation in order to achieve a photochemical surface treatment.

### BRIEF DESCRIPTION OF THE DRAWINGS

The present method and the corresponding device are made more apparent in brief in the following using preferred embodiments with reference to the drawings without the intention of limiting the overall inventive idea.

FIG. 1 shows an example of the setup of the present device as well as its operation;

FIG. 2 shows another example of an embodiment of the present device and its operation; and

FIG. 3 shows a third example of an embodiment of the present device and its operation.

### WAYS TO CARRY OUT THE INV NTION

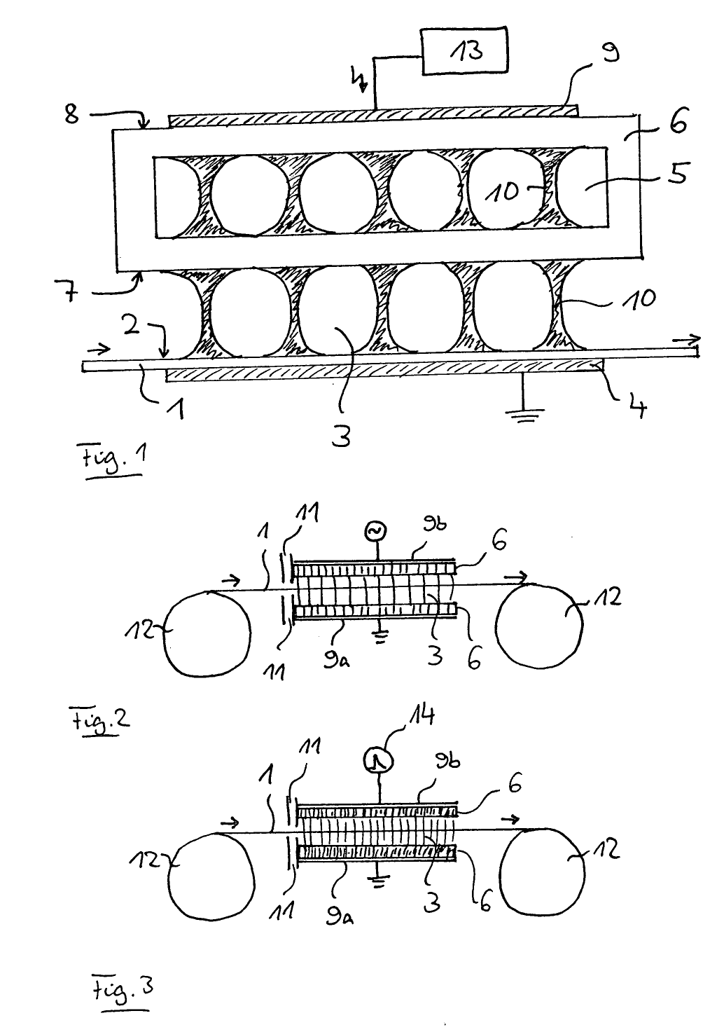

FIG. 1 depicts an example of the embodiment of the present device as well as its manner of operation. The figure shows the discharge region 3 which is formed between a first planar electrode 4 and a chamber 6 made of an UV-permeable dielectric material. A further electrode 9, to which high voltage from a high voltage generator 13 is applied, is placed on side 8 of the chamber 6 facing away from the first electrode 4. This high voltage lies usually in an order of magnitude of about 15 kV and is applied as an alternate voltage with 50 Hz to 200 kHz. Chamber 6 is filled with an excimer noble gas. Between the first side 7 of chamber 6 and the first electrode 4, there is moist air in the discharge region 3. The material to be degerminated in the present instance, in this example a plastic foil 1, is led in arrow direction close to the first electrode 4 through the discharge region 3.

In the surface treatment of plastic foil, the high voltage applied between the two electrodes 4, 9 sparks a barrier discharge in the noble gas inside chamber 6 as well as in the air of the discharge region 3, which is made more apparent in the present representation by the sketched discharge filaments 10. The barrier discharge in the discharge region 3, hereinafter referred to as the first discharge, acts directly on the surface 2 of the plastic foil 1 in such a manner that plasma-chemical cleansing is achieved. The barrier discharge inside chamber 6, hereinafter referred to as the second discharge, leads, due to the selected excimer gas, to a strong UV emission which passes through the UV-permeable dielectric material of side 7 of chamber 6 and acts on the surface 2 of the plastic foil 1 simultaneously with the first barrier discharge so that photochemical cleansing enhances the plasma-chemical cleansing action.

This cascade barrier discharge permits degerminating the surface of the plastic foil 1 more efficiently and faster. For example, measurements showed a 99.999% germ reduction on the surface of a PET foil in less than 2 seconds.

FIG. 2 depicts an embodiment of the present device showing at the edge of the discharge region 3 the additional gas supply lines 11 for introducing gases that enhance the surface treatment process. In this example, a further chamber 6 made of a dielectric material with a UV emitting plasma-excited gas is also placed on the side of the first electrode 9a. The discharge region 3 is located between the two chambers 6 which again border the planar electrodes 9a, 9b. In this example, both chambers 6 are designed identically and are filled with excimer gas. In this arrangement, the foil 1 is impinged on both sides by barrier discharges and UV radiation in such a manner that two-sided surface treatment occurs. During the surface treatment, the foil 1 is led through the discharge region 3 over winding and unwinding reels 12.

Finally, FIG. 3 shows a further development of the device according to FIG. 2 in which high voltage impulses 14 are applied to the electrodes 9a, 9b so that a denser distribution of the filaments inside the gas discharges is achieved. This denser distribution of the filaments increases the uniformity of the surface treatment and improves UV conversion efficiency of the barrier discharge in chamber 6, for example from 30% to 60% in Xe.

List of Reference Numbers

1 object

2 surface of the object

3 discharge region

4 first electrode

5 second electrode

6 chamber

7 first side of the chamber

8 second side of the chamber

9 further electrode

9a first electrode

9b second electrode

10 filaments

11 gas supply lines

12 winding resp. unwinding reels

13 high-voltage generator

14 high-voltage impulses

What is claim d is:

1. A method for treating the surface of objects, in particular made of strip material or of deep-drawn material, in which the to-be-treated surface (2) of the object (1) is subjected in a discharge region (3) filled with a first gas or gas mixture to a barrier discharge generated between a first planar electrode (4) and a second planar electrode (5),
wherein
a plasma-excited second gas or gas mixture emitting UV radiation to which said to-be-treated surface (2) is additionally subjected is provided as said second electrode (5).

2. A method according to claim 1,
wherein
said plasma-excited second gas or gas mixture is under a pressure of at least 100*10² Pa.

3. A method according to claim 1 or 2,
wherein
a gas or gas mixture forming excimers in a plasma-excited state is provided as said plasma-excited second gas or gas mixture.

4. A method according to one of the claims 1 to 3,
wherein
one or a multiplicity of additional gases which enhance the effect of the surface treatment and/or the uniformity of the barrier discharge are introduced into said discharge region (3).

5. A method according to claim 4,
wherein
helium, argon, nitrogen, hydrogen, oxygen, ozone, water gas, water vapor, hydrogen peroxide gas or hydrogen peroxide vapor or a combination of said gases are introduced into said discharge region (3).

6. A method according to one of the claims 1 to 5,
wherein
pulsed voltages are applied to said electrodes (4, 9).

7. A method according to one of the claims 1 to 6,
wherein
a planar first electrode (4) is provided which can be provided metallic or with a dielectric layer on the object side.

8. A method according to claim 7,
wherein
said first electrode (4) and said second electrode (5) are designed in such a large surface manner that when treating the surface of a strip material they expand further in at least one dimension than the width of the strip material.

9. A method according to one of the claims 7 or 8,
wherein
a plurality of first electrodes (4) and second electrodes (5) are placed side by side and/or behind each other in order to treat a large surface region simultaneously.

10. A method according to one of the claims 1 to 9,
wherein
said object (1) is moved through said discharge region during the barrier discharge.

11. A method according to one of the claims 1 to 10 to clean and/or disinfect and/or sterilize and/or activate surfaces, in particular to degerminate or sterilize packing material.

12. A device for treating the surface of flat objects, in particular of strip material or of deep-drawn material,said device having a discharge region (3) which is formed between a first planar electrode (4, 9a) and a chamber (6) filled with a gas, said chamber (6) being made of a dielectric material permeable for UV radiation on at least one first side (7) facing said first electrode (4, 9a),
wherein
said chamber (6) borders on a second side (8) facing away from said first electrode (4, 9a) a further planar electrode (9, 9b) or is closed by the same and is filled with a gas or gas mixture emitting UV radiation in a plasma-excited state.

13. A device according to claim 12,
wherein
the pressure of said gas respectively said gas mixture in said chamber (6) is at least 100*10² Pa.

14. A device according to claim 12 or 13,
wherein
said first electrode (4, 9a) and said further electrode (9, 9b) as well as said first side (7) and said second side (8) of said chamber (6) are designed planar.

15. A device according to claim 12 or 13,
wherein
said first electrode (4, 9a) and said further electrode (9, 9b) as well as said first side (7) and said second side (8) of said chamber (6) are designed three-dimensional, in particular curved.

16. A device according to one of the claims 12 to 15,
wherein,
said discharge region (3) is filled with air, moist air, oxygen or argon or air, moist air, oxygen or argon flow through said discharge region (3).

17. A device according to one of the claims 12 to 16,
wherein
said chamber (6) is filled with a gas or gas mixture which forms excimers in a plasma-excited state, for example Xe or KrCl.

18. A device according to one of the claims 12 to 17,
wherein
said dielectric material of said chamber (6) is quartz glass, in particular a synthetic quartz glass.

19. A device according to one of the claims 12 to 18,
wherein
a chamber (6) made of dielectric material is placed between said discharge region (3) and said first electrode (9a), said chamber (6) being filled with a gas or gas mixture which emits UV radiation in a plasma-excited state.

20. A device according to one of the claims 12 to 19,
wherein
said first electrode (4, 9a) is spaced a distance of between 0.1 to 5 mm from said first side (7) of said chamber (6).

21. A device according to one of the claims 12 to 20,
wherein
said discharge region (3) is open on at least two sides.

22. A method for operating a device according to one of the claims 12-21,
in which between said first electrode (4, 9a) and said further electrode (9, 9b), a sparking voltage is applied which suffices to spark only a discharge in said chamber (6) but does not suffice to spark a discharge in said discharge region (3) so that objects placed in said discharge region (3) are only impinged with UV radiation from said chamber (6).
